(19) United States
(12) Patent Application Publication
(10) Pub. No.: US 2016/0160181 A1
(43) Pub. Date: Jun. 9, 2016

(54) PROCESSES FOR PRODUCING EXOSOMES IN REDUCED OXYGEN CULTURE CONDITIONS

(71) Applicant: Capricor Thera[eitocs, Inc., Beverly Hills, CA (US)

(72) Inventors: Michelle KREKE, Marina Del Rey, CA (US); Rachel SMITH, Cary, NC (US); Ahmed IBRAHIM, Los Angeles, CA (US)

(21) Appl. No.: 14/958,784

(22) Filed: Dec. 3, 2015

Related U.S. Application Data
(60) Provisional application No. 62/086,742, filed on Dec. 3, 2014.

(51) Int. Cl. C12N 5/077; C12N 15/113

(52) U.S. Cl. CPC C12N 5/0657; C12N 15/113; C12N 2500/02; C12N 2310/141; C12N 2330/10; C12N 2320/30

(57) ABSTRACT

The invention encompasses methods for generating exosomes comprising culturing cells in less than 20% oxygen for at least 2 days and harvesting exosomes from the cells. The invention further encompasses exosome preparations generated from cells cultured in less than 20% oxygen for at least 2 days.

### BACKGROUND OF THE INVENTION

Exosomes are cell-derived vesicles. Hong et al., PLoS ONE 9(8): e103310. doi:10.1371/journal.pone.0103310. They are found in biological fluids, such as urine, plasma, and ascites. Id. Exosomes are generated by inward budding of endosomal multivesicular bodies. Id. The cargo of exosomes includes proteins/glycoproteins expressed on the cell membrane as well as molecules and soluble factors present in the cytosol of parental cells. Id. Exosomes normally have diameters ranging from 40-100 nm. Zhang et al., Oncology Letters 8: 1701-1706, 2014. Exosomes contain special proteins, lipids, RNA and micro-RNAs. Id.

Exosomes produced from cardiosphere-derived cells enhance angiogenesis and promote cardiomyocyte survival and proliferation. Ibrahim et al., 2014 May 8; 2(5):606-19, which is hereby incorporated by reference. Exosomes produced from cardiosphere-derived cells are enriched in miR146a.

The leading cause of death in the US remains heart disease. Kochanek et al., Natl Vital Stat Rep 2011; 60:1-116. Adjusting for an aging population, the global incidence and mortality from ischemic heart disease is decreasing due current standard of care improvements in major adverse cardiac events (MACE). Moran et al., Circulation. 2014; 129:1493-1501. However, the result is an increasing number of heart attack survivors and disability years due to nonfatal ischemic heart disease, which contributes greatly to the overall global economic burden of ischemic heart disease. Id. This suggests a need now to shift from MACE improvements over current standard of care to improvements in quality of life, fitness and vitality for the surviving patients with chronic angina and heart failure. Id.

Cardiosphere-derived cells (CDCs) are cells obtained from heat samples with regenerative and immunomodulatory capabilities. Therapeutic capabilities of CDCs are being evaluated in clinical testing. CDCs administered after a myocardial infarction (MI) in two clinical trials (CADUCEUS and ALLSTAR) have been shown to be safe and effective in reducing scar size and increasing viable myocardium. Exosomes represent a next generation therapeutic platform for regenerative medicine. These nano-sized extracellular membranous vesicles are potent delivery vehicles for functional messenger RNA (mRNA), microRNA (miRNA) and DNA molecules as well as proteins and growing evidence suggests they can impart similar therapeutic benefits as the producer cells. CDC-derived exosomes have been shown to recapitulate the effects of CDCs now in numerous preclinical models. de Couto et al., Circulation. 2014; 130; Ibrahim et al., Stem Cell Reports. 2014; 2:606-619; Tseliou et al., Circulation. 2014; 130. Research has shown that CDCs secrete exosomes containing particular miRNAs that, limit fibrosis, modulate immune response, stimulate cardiomyocyte proliferation, spur angiogenesis, and improve functional recovery in MI models. The totality of the preclinical data demonstrate that exosomes represent a required, secreted active pharmaceutical ingredient (API) for CDCs' primary mechanism of action (MoA).

CDCs and their isolated exosomes hold great therapeutic potential to relieve this global burden of heart disease. CDCs in the Phase 1 CADUCEUS and ALLSTAR clinical trials have been shown to reduce scar size and increase myocardial tissue viability (see FIG. 1). Malliaras et al., J Am Coll Cardiol. 2014; 63:110-122; Makkar R et al., Lancet. 2012; 379:895-904; Makkar et al., Journal of the American College of Cardiology. 2014; 64.

CADUCEUS was the first clinical trial to observe increases in viable myocardium suggesting therapeutic regeneration. Makkar R et al., Lancet. 2012; 379:895-904. The ongoing ALLSTAR Phase 2 trial with a 5 year sub-study will assess quality of life metrics and impact on hospitalization and mortality.

Nano-sized exosomes have major manufacturing and toxicology advantages over cells such as the ability to increase sterility assurance in the process using microbial retentive filters (e.g. ≦0.22 μm filters). Exosomes as non-living present potentially lower risks for adverse tumorigenic and immunogenic responses due to their very nature as non-living. Exosomes also certainly possess more flexibility in terms of stable drug storage temperature options compared to cells (e.g. room and cold temperatures vs. liquid nitrogen). The current research process for generating CDC-exosomes involves first seeding and growth of CDCs to confluence in fetal bovine serum (FBS)-containing conditions. For exosome production, the confluent layer of CDCs are washed and cultured under serum-free (free from FBS-exosomes), normoxic (20% O₂) conditions for 15 days. Exosomes are then isolated from thawed, conditioned media (containing exosomes) using a precipitation method (intended for research use only), and formulated in base serum-free medium (a research grade reagent).

CDC exosomes are capable of improving cardiac function, stimulating angiogenesis and cardiomyocyte proliferation, modulating inflammatory process, and inhibiting cardiomyocyte apoptosis but not normal human dermal fibroblasts (NHDF) derived exosomes. When exosome secretion is inhibited using GW4869, the cardiac functional benefits of CDCs were diminished.

CDC exosome microRNA composition was characterized with a miRNA array. It was found that miR-210 and miR-146a were up-regulated in CDC exosomes in comparison to NHDF exosomes.

miR-210 is key player of the cellular response to hypoxia and capable of modulating cell survival and mitochondrial metabolism of both endothelial cells and cardiomyocytes. In addition, miR-210 has been shown to play a role in T cell differentiation (Ref: Nat Immunol (2014). 15, 393-401). Hypoxia-inducible factor 1-alpha (HIF1α) directly binds to a hypoxia responsive element (HRE) on the proximal miR-210 promoter. HIF1a has been identified as a target of miR-210, suggesting a negative feedback by miR-210 in inhibiting HIF-1a expression (Ref: Nat Immunol (2014). 15, 393-401). The downstream targets of the HIF1α pathway is stromal cell-derived factor-1 (SDF-1) and vascular endothelial growth factor (VEGF). miR-210 is part of the key CDC exosome miRNA quantitative polymerase chain reaction (qPCR) panel to evaluate process parameters.

miR-146a is a pivotal immune regulatory molecule in various diseases and is induced upon the activation of toll-like receptor 4 (TLR4) in a nuclear factor kappa-light-chain-enhancer of activated B cells (NF-κB)-dependent signaling pathway which leads to the down regulation of interleukin 1 (IL-1) receptor-associated kinase 1 (IRAK1). Among the molecular targets of miR-146a is the CXCR4 pathway, which is a seven transmembrane G-protein coupled receptor of SDF-1 involved the innate and adaptive immune response. miR-146a is part of the key CDC exosome miRNA quantitative polymerase chain reaction (qPCR) panel to evaluate process parameters.

There is a need in the art for better exosome preparations, particularly for clinical use, having varied protein and RNA constituents, and that can be produced in a shorter period of time. The invention fulfills this need in the art.

### BRIEF SUMMARY OF THE INVENTION

The invention encompasses methods for generating exosomes comprising culturing cells in less than 20% oxygen for at least 2 days and harvesting exosomes from the cells. The invention further encompasses an exosome preparation generated from cells cultured in less than 20% oxygen for at least 2 days.

Preferably, the cells are cultured for at least 5 days, particularly 5-15 days, 5-10 days, 10-15 days, or at least 15 days.

Preferably, the cells are cultured in 2-8% 3-7%, 4-6%, or 4.5-5.5% oxygen.

In one embodiment, the cells are cardiosphere-derived cells (CDCs).

In one embodiment, the cells are passaged for at least 5 passages.

In one embodiment, the exosome preparation comprises less than 5% polyethylene glycol.

In one embodiment, the exosomes are purified using polyethylene glycol. In one embodiment, the exosomes are purified using ultrafiltration. In one embodiment, polyethylene glycol is added to the exosomes after purification.

### BRIEF DESCRIPTION OF THE DRAWINGS

FIGS. 1A and 1B depict CDC -derived exosome size and concentration in condition media and after ultrafiltration (UFC) as quantified by Brownian motion using the Nanosight nanoparticle tracking analysis.

FIGS. 2A and 2B depict the change in CDC-derived exosome size and resulting concentration after PEG (ExoQuick) (A) precipitation or antibody crosslinking (B) as quantified by Brownian motion using the Nanosight nanoparticle tracking analysis.

FIG. 3 depicts total protein quantity in CDC-derived exosomes isolated by various PEG precipitation solutions, ExoQuick and ultrafiltration as quantified by DC protein assay.

FIGS. 4A to 4D depict CDC-derived exosome stability at various temperature conditions (4 to −80° C.) after ultrafiltration (UFC) isolation as quantified by Brownian motion using the Nanosight nanoparticle tracking analysis.

FIG. 5 depicts increased overall quantity of CDC-derived exosomes in conditioned media and isolated using ultrafiltration (UFC) with/or without 0.22 μm microbial reduction filter after 5 or 15 days of culture as quantified by Brownian motion using the Nanosight nanoparticle tracking analysis.

FIG. 6 depicts increased total protein quantity in CDC-derived exosomes in PEG precipitated (Exoquick) and with ultrafiltration (UFC) preparations after 5 or 15 days of culture as quantified by DC protein assay.

FIG. 7 depicts increased total protein concentration in CDC-derived exosomes with increasing CDC passage number after 5 or 15 days of culture as quantified by DC protein assay.

FIG. 8 depicts overall increase in total number of CDC-derived exosomes at physiologic oxygen concentrations (5% O₂) from both 5 and 15 day cultures as quantified by Brownian motion using the Nanosight nanoparticle tracking analysis.

FIG. 9 depicts overall increased total protein quantity in CDC-derived exosomes with physiologic oxygen concentrations (5% O₂) and ultrafiltration (UFC) isolation from 5 and 15 day cultures as quantified by DC protein assay.

FIG. 10 depicts overall increased total RNA quantity in CDC-derived exosomes with ultrafiltration (UFC) isolations and especially at physiologic oxygen concentrations (5% O₂) and 15 days of culture as quantified by NanoDrop spectrophotometer at 260 nm absorbance.

FIG. 11 depicts increased total RNA quantity in CDC-derived exosomes with 25% PEG precipitation over 50% and 75% PEG solutions and Exoquick as quantified by Qubit® fluorometer using RNA assay kit with 630/680 nm absorbance.

FIG. 12 depicts up-regulated miR-146A expression in CDC-derived exosomes relative to U6 housing gene and negative control fibroblast (NHDF) derived exosomes as quantified by quantitative polymerase chain reaction (qPCR) using TaqMan® MicroRNA assay.

FIG. 13 depicts up-regulated miR-210 expression in CDC-derived exosomes relative to U6 housing gene and negative control fibroblast (NHDF) derived exosomes as quantified by quantitative polymerase chain reaction (qPCR) using TaqMan® MicroRNA assay.

FIG. 14 depicts similar up-regulated miR-146A expression in CDC-derived exosomes from 15 day cultures (5% O₂) and isolated with various PEG precipitation solutions relative to U6 housing gene and negative control fibroblast (NHDF) derived exosomes as quantified by quantitative polymerase chain reaction (qPCR) using TaqMan® MicroRNA assay.

FIG. 15 depicts similar up-regulated miR-210 expression in CDC-derived exosomes from 15 day cultures and isolated with various PEG precipitation solutions relative to U6 housing gene and negative control fibroblast (NHDF) derived exosomes as quantified by quantitative polymerase chain reaction (qPCR) using TaqMan® MicroRNA assay.

FIG. 16 depicts up-regulated miR-146A expression in CDC-derived exosomes from 15 day cultures and lower oxygen concentrations (2% and 5% O₂) relative to U6 housing gene and negative control fibroblast (NHDF) derived exosomes as quantified by quantitative polymerase chain reaction (qPCR) using TaqMan® MicroRNA assay.

FIG. 17 depicts up-regulated miR-210 expression in CDC-derived exosomes from 15 day cultures and lower oxygen concentrations (2% and 5% O₂) relative to U6 housing gene and negative control fibroblast (NHDF) derived exosomes as quantified by quantitative polymerase chain reaction (qPCR) using TaqMan® MicroRNA assay.

FIG. 18 depicts similar protein and upregulated miR-210 and miR-146A expression in CDC-derived exosomes from 15 day cultures isolated and concentrated with either ExoQuick or Ultrafiltration by centrifugation.

FIGS. 19A and 19B depicts similar particle concentration and size of CDC-derived exosome isolated from 14 day cultures isolated and concentrated with 2 kDa to 10 kDa ultrafiltration membranes. A slight decrease in exosome concentration was observed with larger 30 kDa ultrafiltration membranes.

FIG. 20 depicts CDC-derived exosomes from 15 day cultures isolated and concentrated with 2 kDA to 30 kDa ultrafiltration membranes showed similar upregulated miR-146A and miR-210 expression.

FIGS. 21A to 21C depicts similar protein and upregulated miR-210 and miR-146A expression in CDC-derived exosomes from 15 day cultures isolated, concentrated and filter sterilized compared to no filter (n/a). With the exosome average size −150 nm and typically less than 200 nm, they can be filter sterilized.

### DETAILED DESCRIPTION OF THE INVENTION

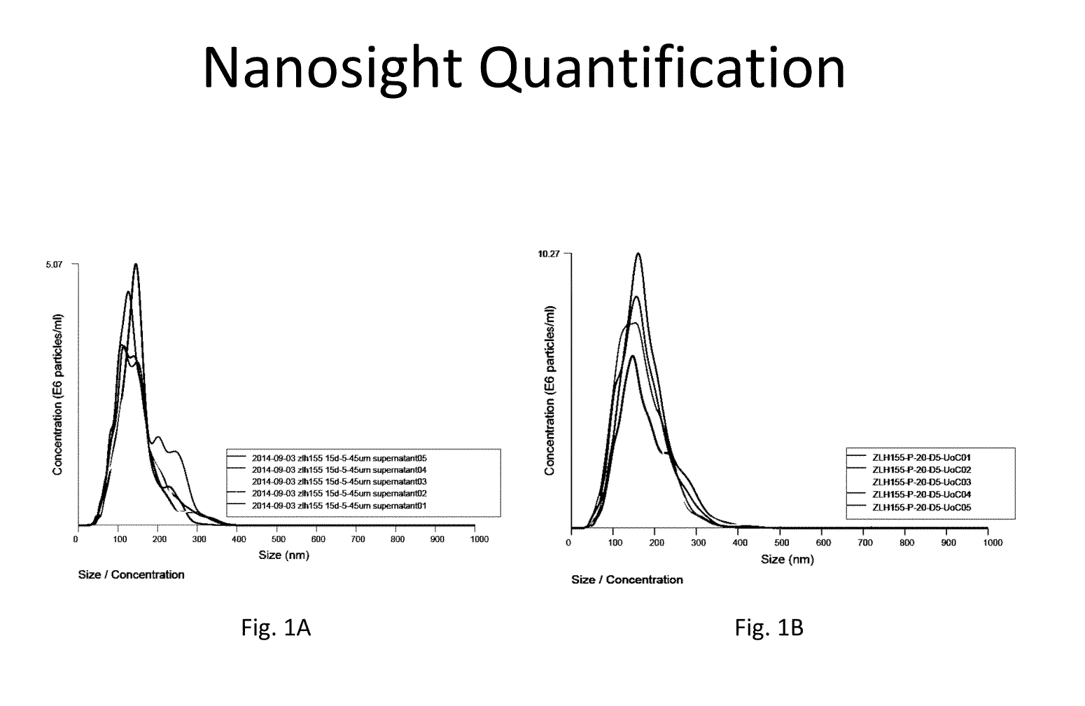

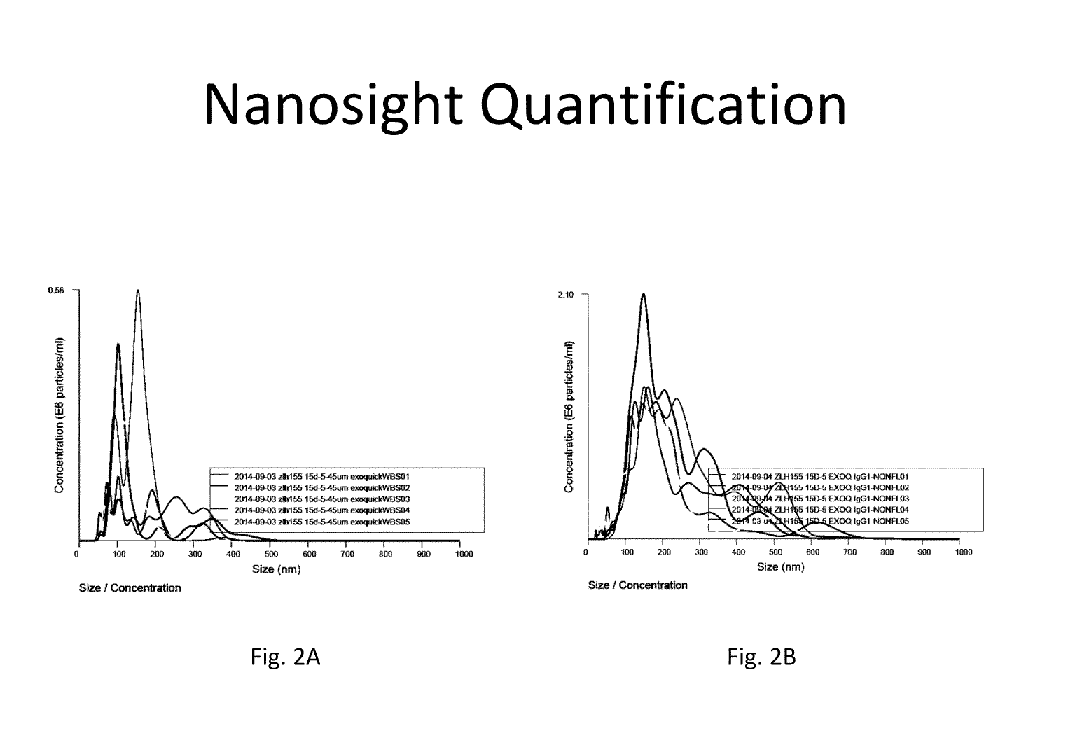

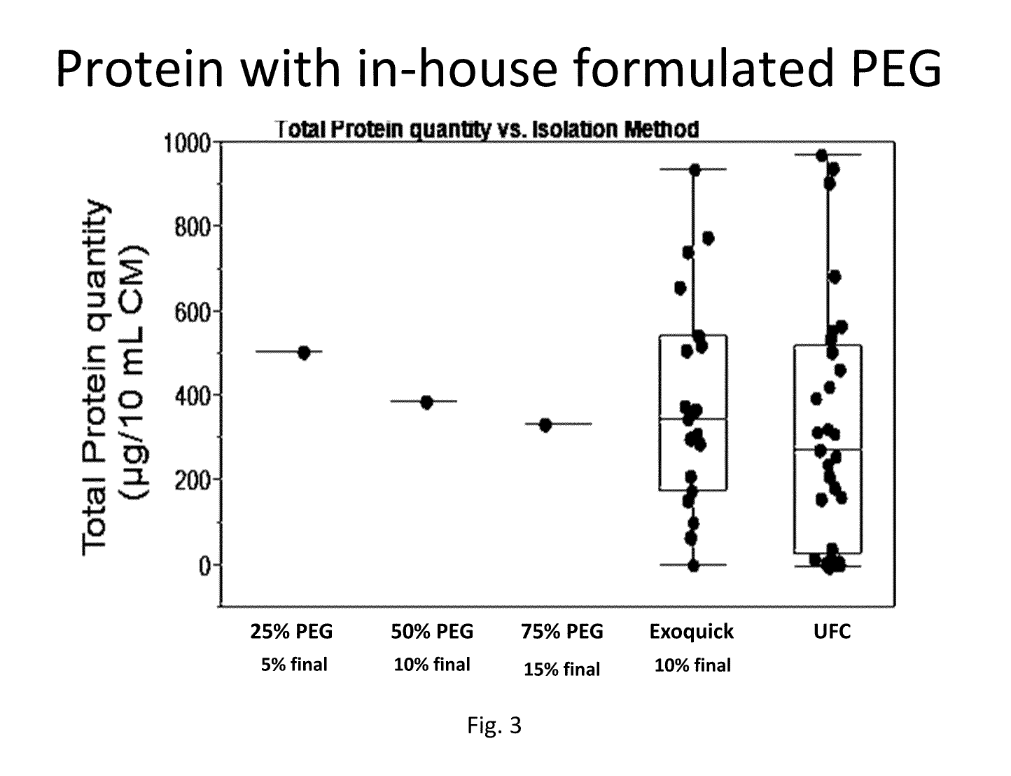

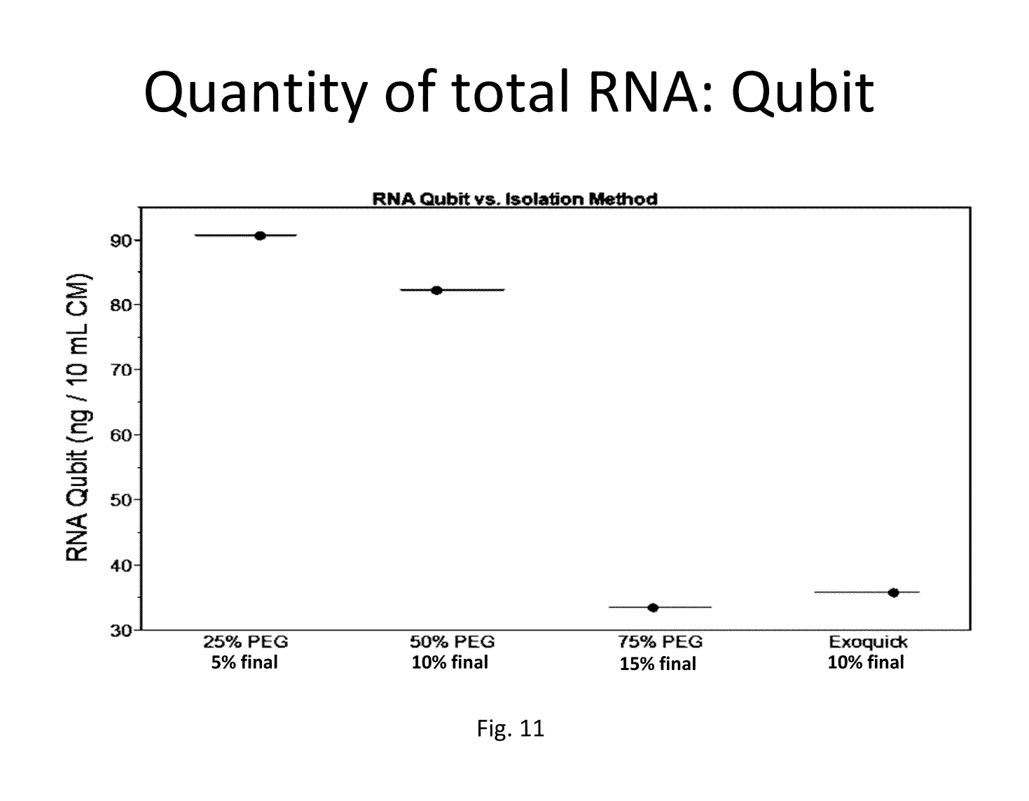

The size and quantity of exosomes produced from primary cells cultured in under standard laboratory conditions of approximately 20% oxygen was determined. (FIG. 1.) The use of PEG was shown to generate aggregates that interfered with quantitation by Nanosight. (FIG. 2.) Ultrafiltration did not generate these aggregates and allowed accurate quantitation. By analyzing total protein quantity in exosomes, it was shown that a variety of PEG preparations and ultrafiltration could be used to prepare exosomes. (FIG. 3 and FIG. 11.)

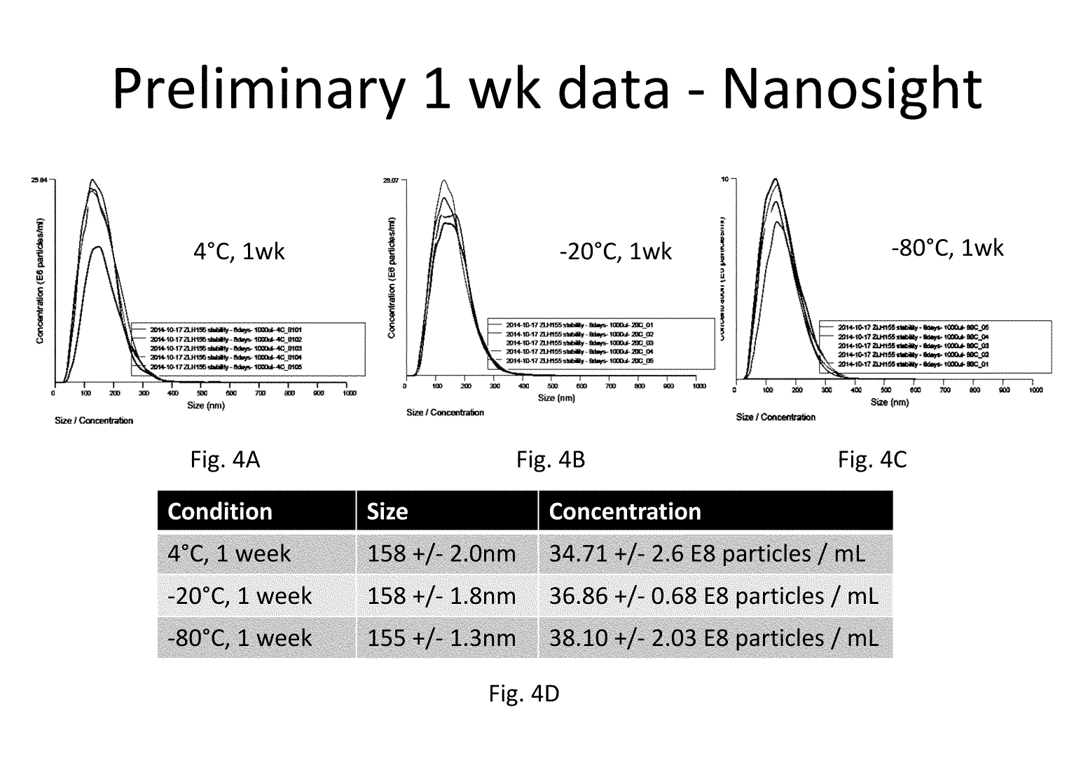

The effect of storing exosomes at various temperatures was examined. (FIG. 4.)

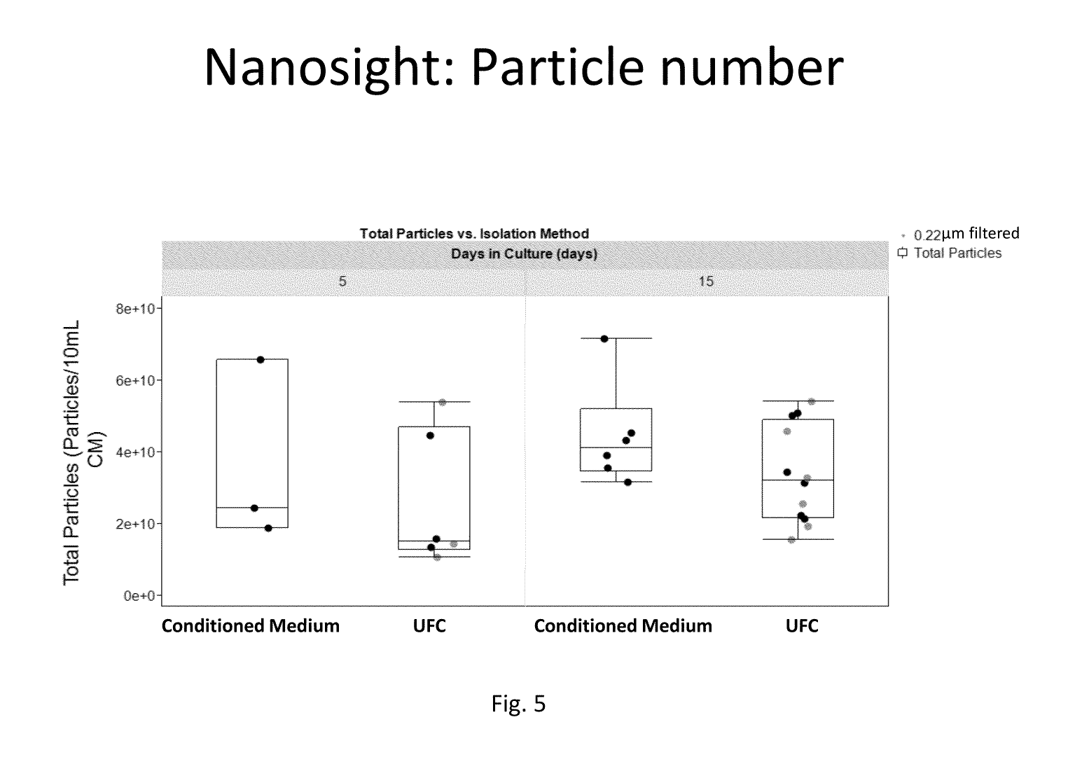

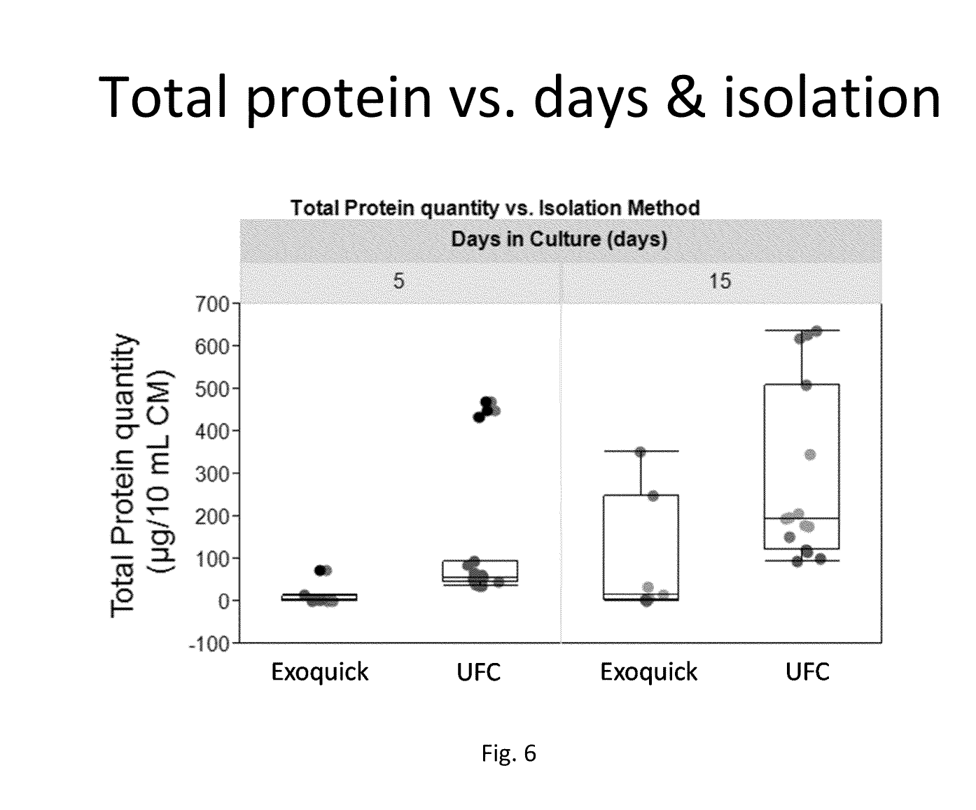

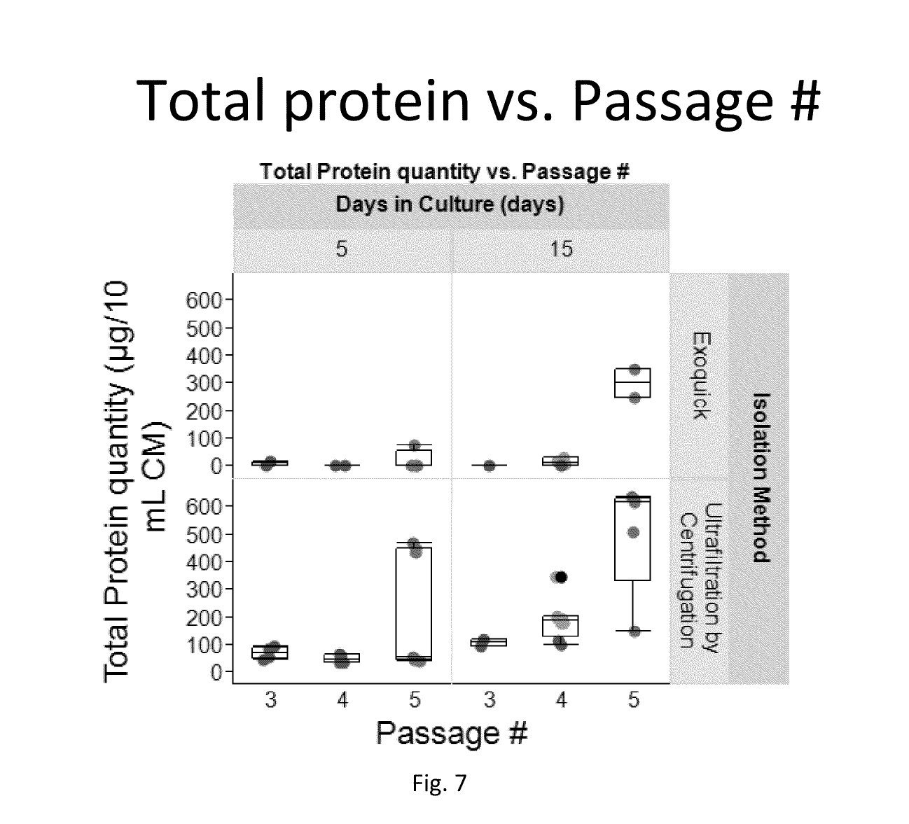

Since exosomes have been prepared from cells using 15 day cultures in serum free medium (Ibrahim 2014), exosome preparations from 5 and 15 day cultures were compared. (FIG. 5.) 15 day cultures showed higher exosome yields. 15 day cultures also showed higher total protein quantity than 5 day cultures. (FIG. 6.) Unexpectedly, exosome preparations from passage 4 and passage 5 cells showed higher total protein quantities than exosome preparations from passage 3 cells. (FIG. 7.)

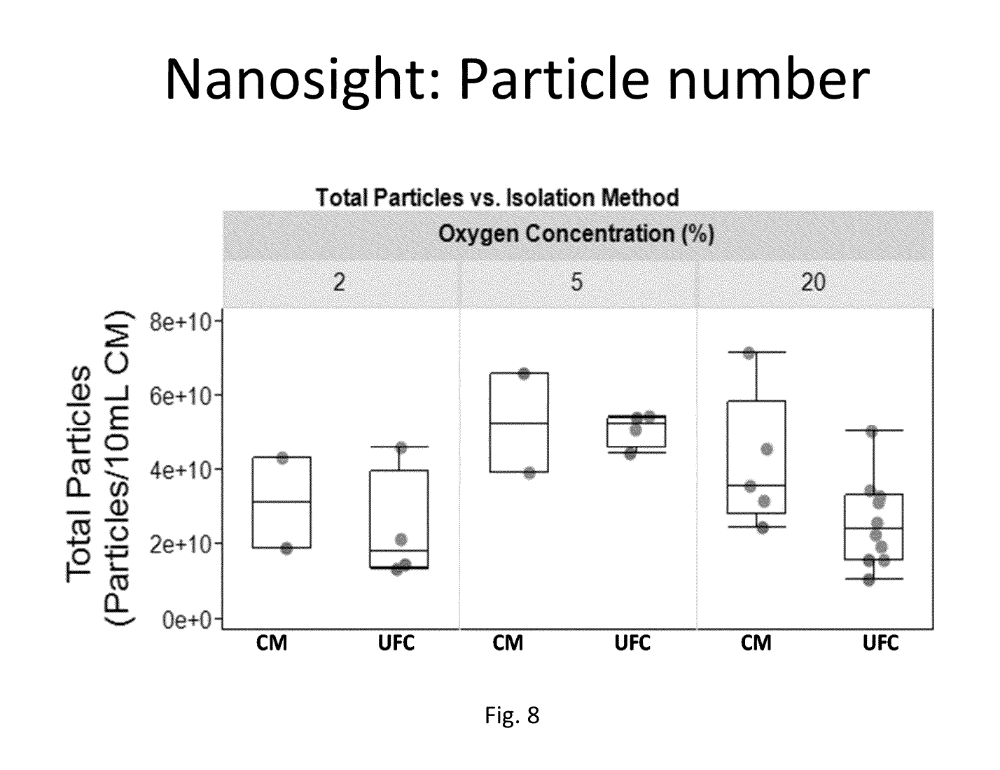

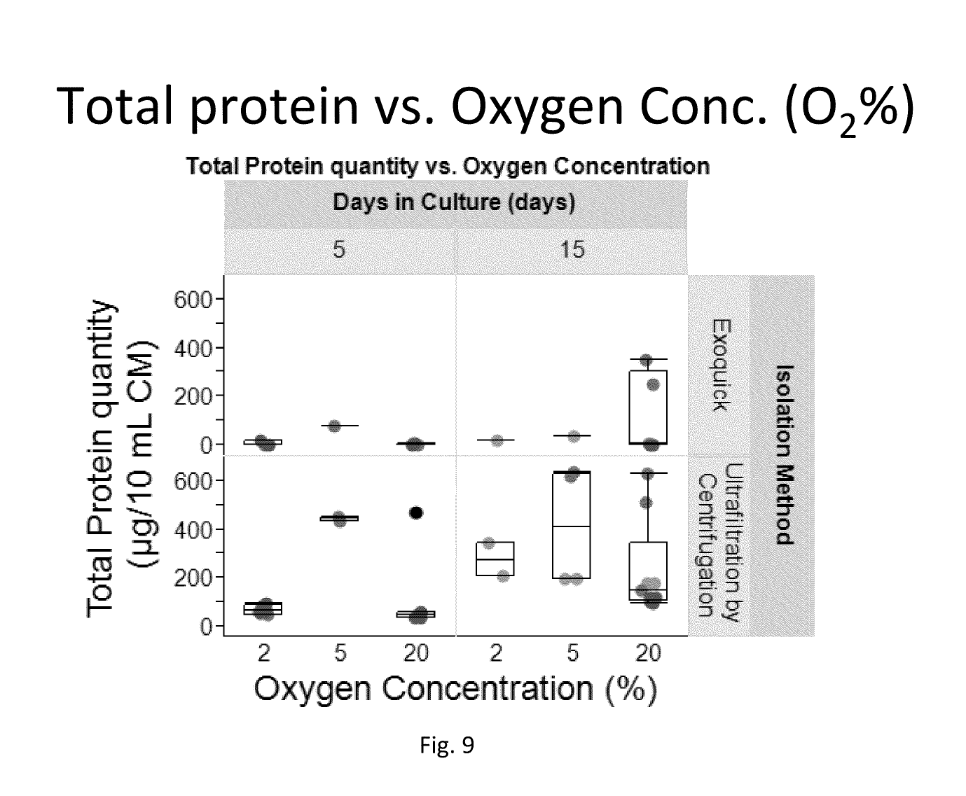

The effect of oxygen concentration on exosome yield was examined. Unexpectedly, lowering the oxygen level from 20% to 5% resulted in a substantial increase in the number of exosomes. (FIG. 8.) Similarly, lowering the oxygen level from 20% to 5% also resulted in a substantial increase in the quantity of total protein associated with exosomes at both day 5 and day 15. (FIG. 9.) Total protein levels at day 5 were even higher than day 15 levels.

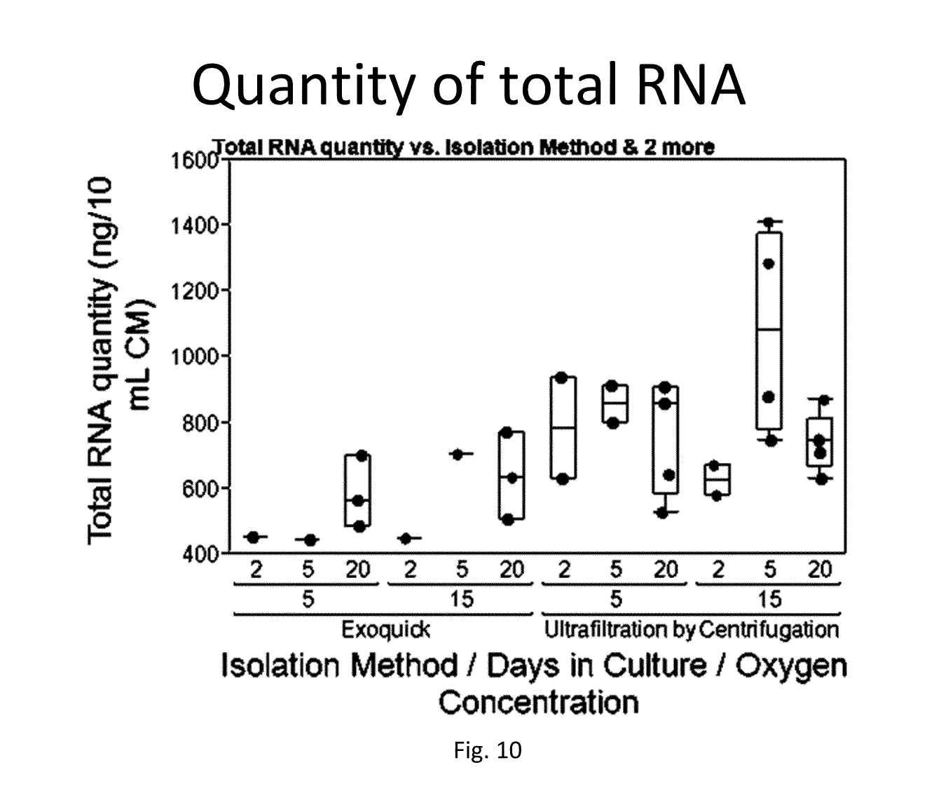

Lowering the oxygen level from 20% to 5% resulted in a substantial increase in the quantity of RNA associated with exosomes at day 15. (FIG. 10.) The quantity of RNA associated with exosomes at day 5 was similar with 20% and 5%

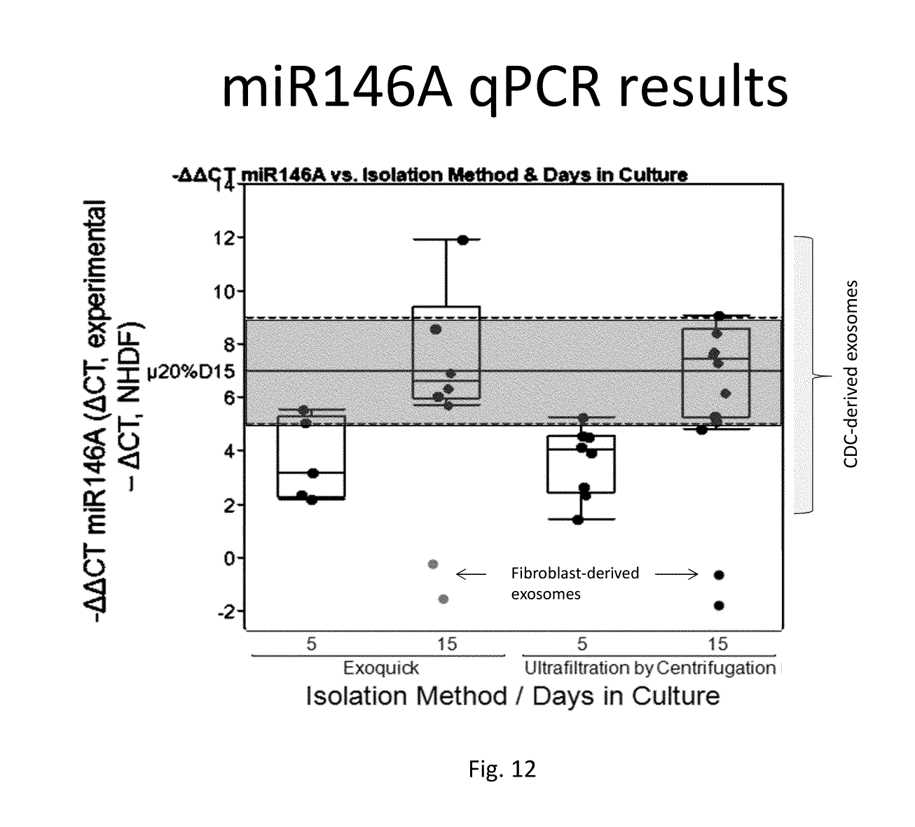

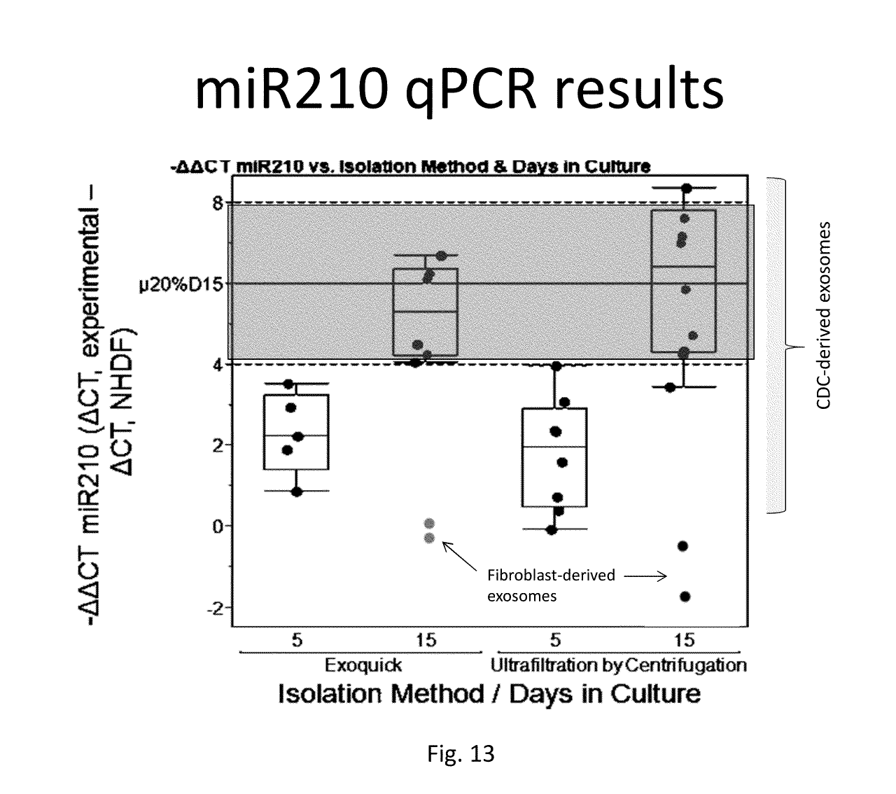

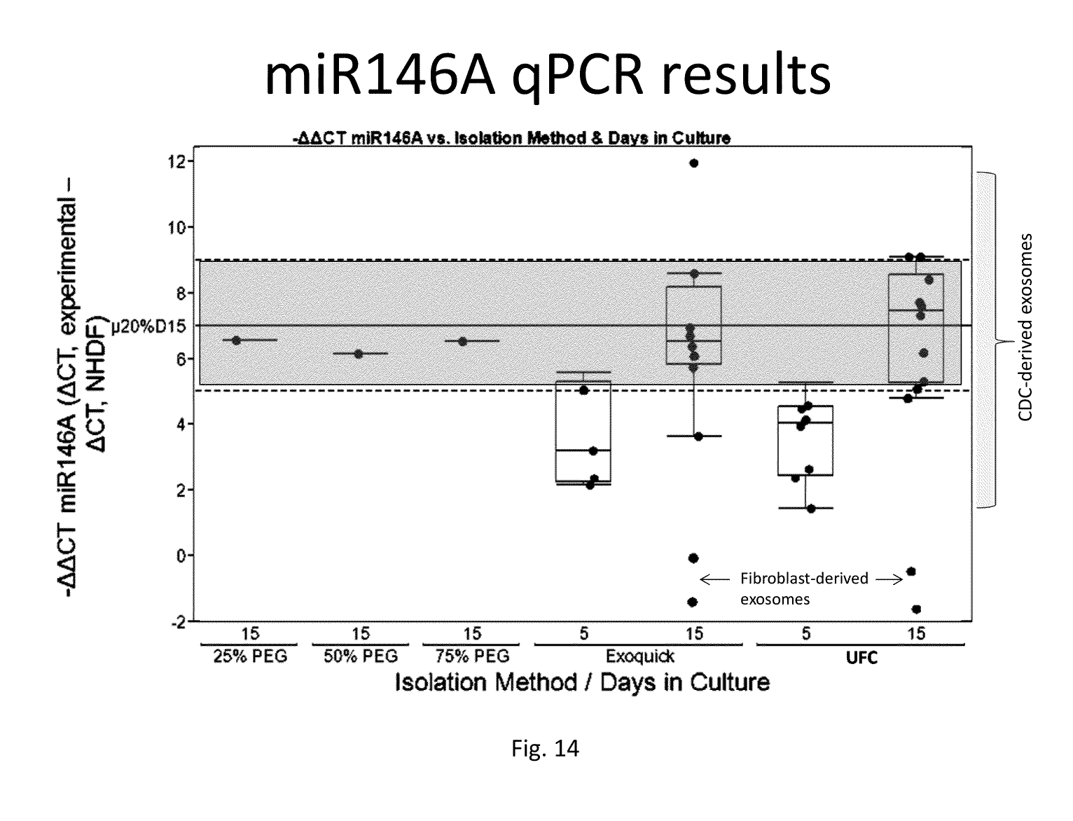

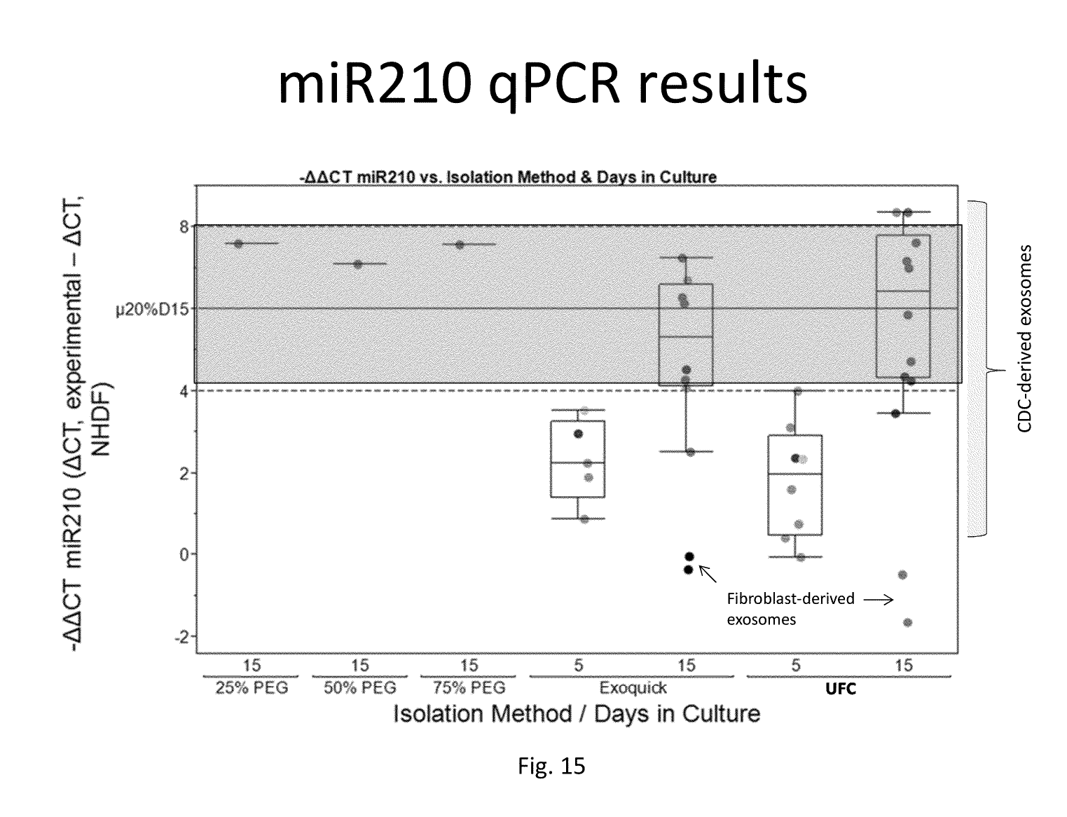

miR-146A and miR-210 RNA levels were examined from day 5 and 15 exosome preparations in 20% oxygen. (FIG. 12 and FIG. 13.) The amount of miR-146A and miR-210 RNA was lower at day 5 than at day 15. (FIG. 14 and FIG. 15.) Alternative exosome preparation procedures gave similar results.

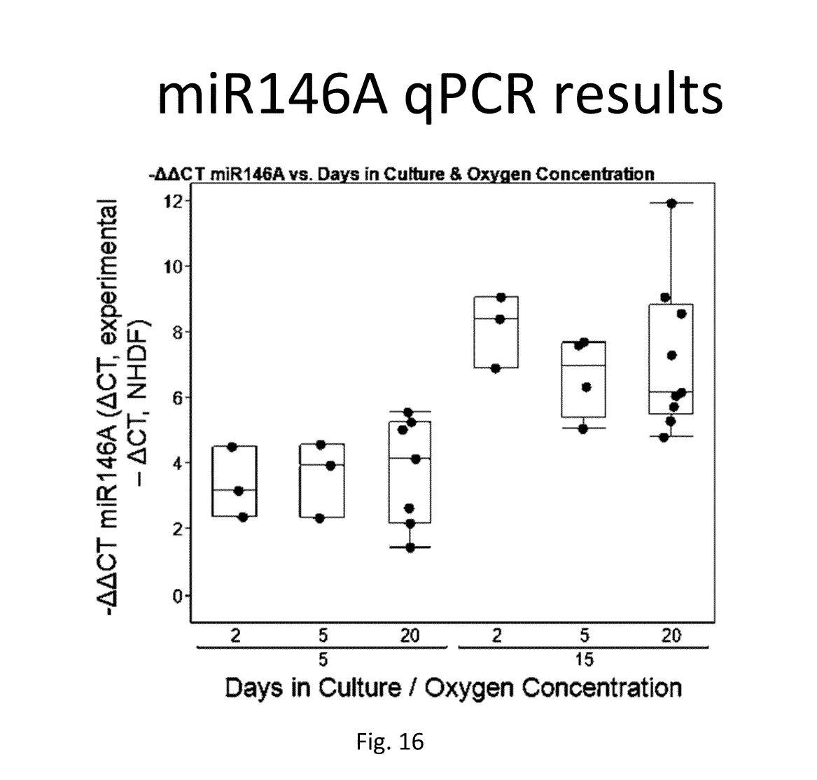

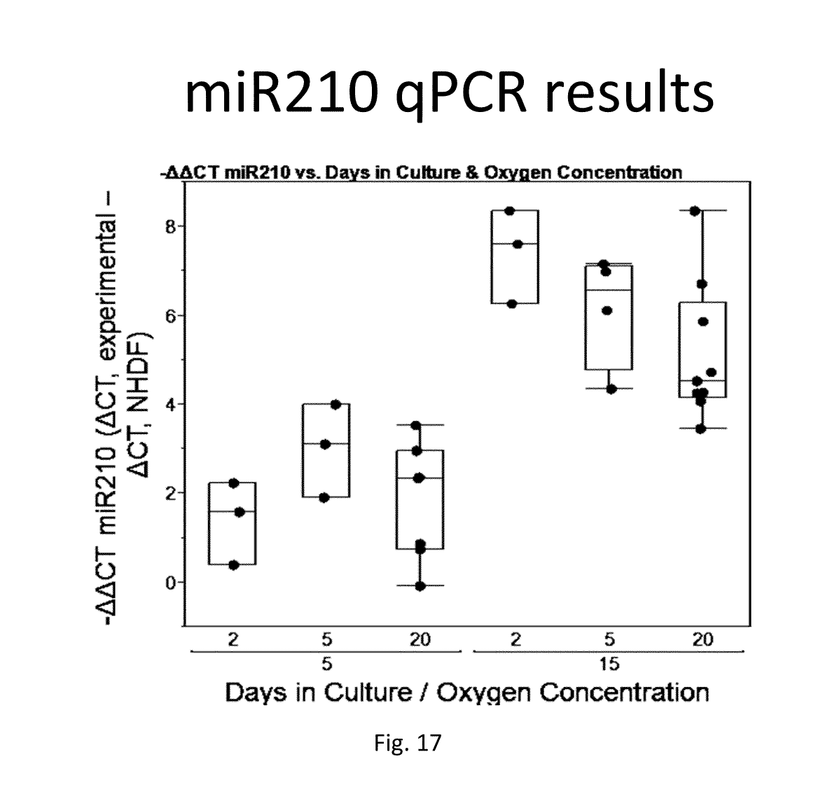

Lowering the oxygen level from 20% to 5% or 2% resulted in a substantial increase in the quantity of miR-146A RNA associated with exosomes at day 15. (FIG. 16.) The quantity of miR-210 RNA associated with exosomes at day 15 was highest with 2% oxygen. (FIG. 17.) The quantity of miR-146A RNA associated with exosomes at day 5 was similar with 20%, 5%, and 2% oxygen.

Lowering the oxygen level from 20% to 5% or 2% resulted in a substantial increase in the quantity of miR-210 RNA associated with exosomes at days 5 and 15.

The quantity of miR-210 RNA associated with exosomes at day 15 was highest with 2% oxygen.

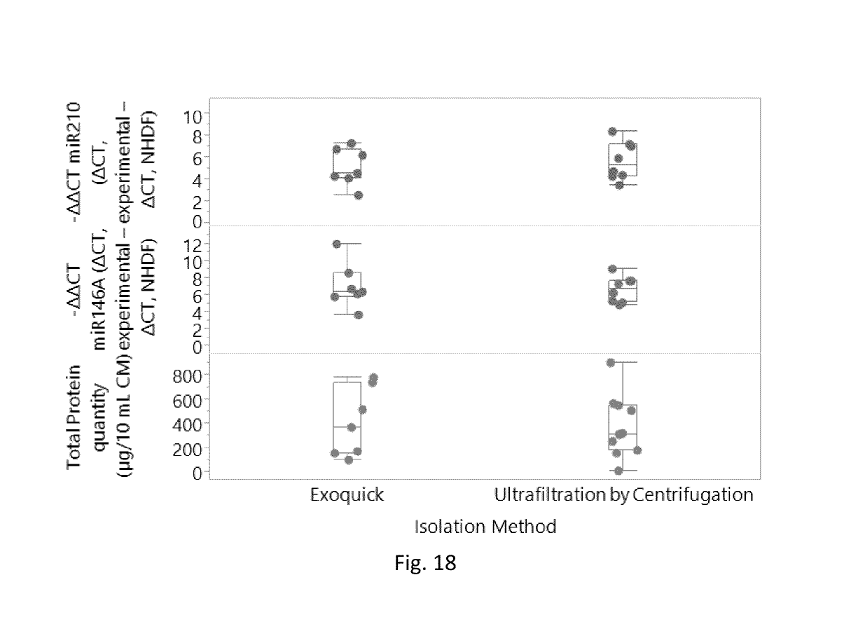

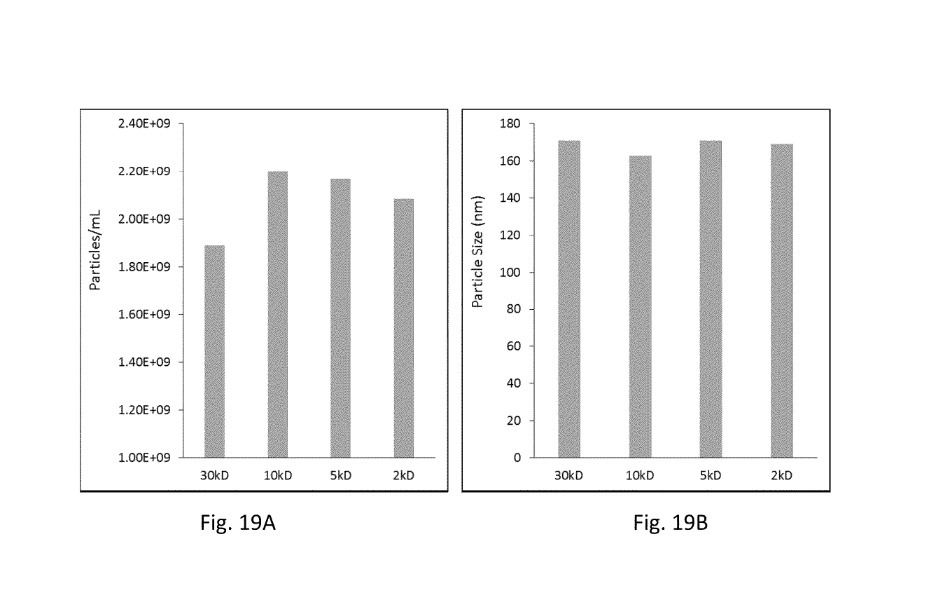

CDC-derived exosomes from 15 day cultures isolated and concentrated with either ExoQuick or Ultrafiltration by centrifugation showed similar protein and upregulated miR-210 and miR-146A expression. (FIG. 18.)

CDC-derived exosome isolated from 15 day cultures isolated and concentrated with 2 kDa to 10 kDa ultrafiltration membranes showed similar particle concentration and size; a slight decrease in exosome concentration was observed with larger 30 kDa. (FIG. 19.)

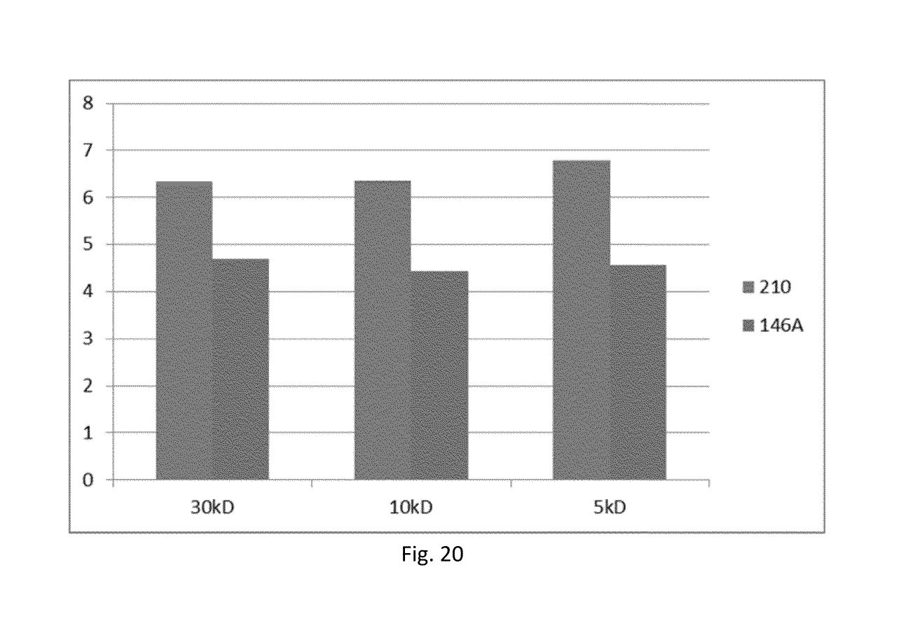

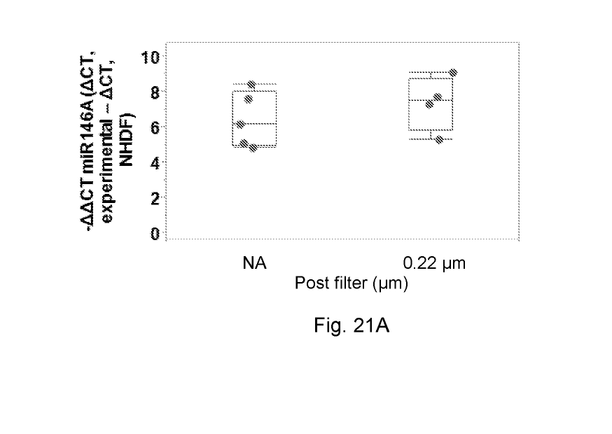

CDC-derived exosomes from 15 day cultures isolated and concentrated with 2 kDA to 30 kDa ultrafiltration membranes showed similar upregulated miR-146A and miR-210 expression. (FIG. 20.)

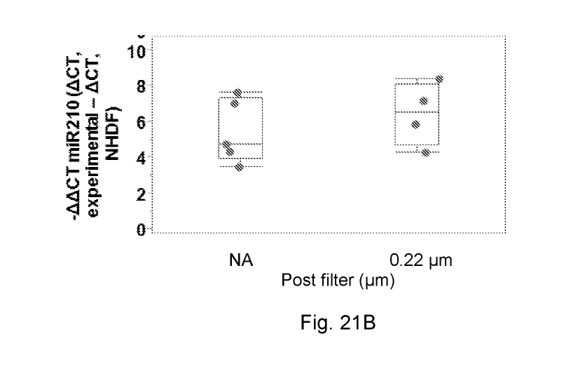

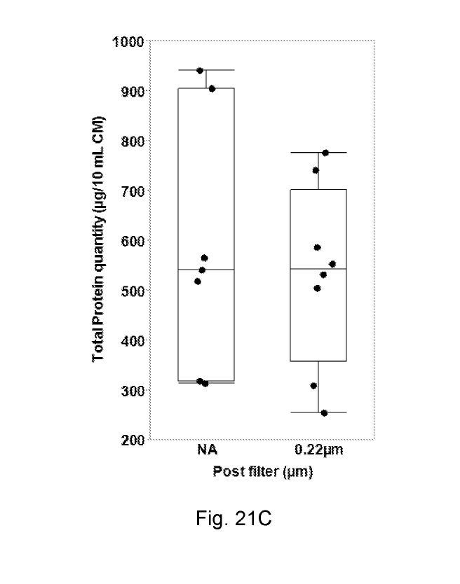

CDC-derived exosomes from 15 day cultures isolated, concentrated and filter sterilized compared to no filter (n/a) showed similar protein and upregulated miR-210 and miR-146A expression. (FIG. 21.) Thus, with the exosome average size ˜150 nm and typically less than 200 nm, they can be filter sterilized.

Thus, reducing the oxygen concentration during culture results in alterations in the quantity and composition of exosomes harvested from these cells. This allows for the generation of exosomes with preferred qualities. The invention encompasses these exosome preparations and methods for producing them.

Methods for Generating Exosomes

The invention encompasses methods for producing exosomes comprising culturing cells. The invention encompasses methods for generating exosomes comprising culturing cells in less than 20% oxygen for at least 2 days and harvesting exosomes from the cells.

Preferably, the cell culture comprises at least 10⁶, 10⁷, 10⁸, 10⁹, 10¹⁰, 10¹¹, or 10¹² cells.

Preferably, the cells are primary cells. The primary cells can be at least at passage number 2, 3, 4, 5, 6, 7, 8, 9, or 10. Immortalized cells are also encompassed by the invention.

Preferably, the cells are human cells. A particularly preferred cell type is cardiosphere-derived cells (CDCs). Other preferred cell types are cardiac tissue derived stem cells, adipose tissue derived stem cells, neural tissue derived stem cells and other tissue derived stem cells.

In one embodiment, the cells can be grown under routine culture conditions, for example, 20% O₂ at 37° C. in IMDM with 20% fetal bovine serum (FBS) and pen/strep by seeding 10⁶ cells per T175 flask.

In various embodiments, the oxygen concentration is 1-2% 2-3%, 4-5%, 5-6%, 6-7%, 7-8%, 8-9%, 9-10%, 10-11%, 11-12%, 12-13%, 13-14%, 14-15%, 15-16%, 17-18%, or 18-19%. Preferably, the oxygen concentration is 2-8%, 3-7% oxygen, 4-6% oxygen, or 4.5-5.5% oxygen.

The cells can be cultured for at least 2, 3, 4, 5, 6, 7, 8, 9, 10, 11, 12, 13, 14, 15, 16, 17, 18, 19, or 20 days. Preferably, the cells are cultured for 5-15 days, 5-10 days, or 10-15 days.

Preferably, the cell culture comprises an insulin supplement and/or chemically defined lipid and cholesterol lipid concentrates.

Exosome Preparations

The invention encompasses exosome preparations generated from the cell cultures of the invention. In various embodiments, the exosome preparation contains exosomes of 50 nm to 250 nm in diameter. Preferably, at least 25%, 50%, 65%, 75%, 80%, 85%, 90%, or 95% of the exosomes are at least 50 nm, 60 nm, 70 nm, 80 nm, 90 nm, 100 nm, 110 nm, 120 nm, 130 nm, 140 nm, 150 nm, or 160 nm. Preferably, at least 25%, 50%, 65%, 75%, 80%, 85%, 90%, or 95% of the exosomes are less than 250 nm, 240 nm, 230 nm, 220 nm, 210 nm, 200 nm, 190 nm, 180 nm, 170 nm, 160 nm, 150 nm, or 140 nm in diameter. Thus, the invention includes exosome preparations wherein at least 25%, 50%, 65%, 75%, 80%, 85%, 90%, or 95% of the exosomes are between 50 nm to 250 nm in diameter, 60 nm to 250 nm in diameter, 60 nm to 240 nm in diameter, 50 nm to 240 nm in diameter, etc.

In some embodiments, the exosome preparation contains at least 10⁵, 5×10⁵, 10⁶, 5×10⁶, 10⁷, 5×10⁷, 10⁸, 5×10⁸, 10⁹, 5×10⁹, 10¹⁰, 5×10¹⁰, 10¹¹, 5×10¹¹, or 10¹², or 5×10¹² exosomes. In some embodiments, the exosome preparation contains between 10⁵, 10⁶, 10⁷, 10⁸, 10⁹, 10¹⁰, or 10¹¹ to 10⁶, 10⁷, 10⁸, 10⁹, 10¹⁰, 10¹¹, or 10¹², etc. exosomes.

In some embodiments, the exosomes preparation includes one or more exosomes containing microRNAs. In various embodiments, these microRNAs can include miR-146A and/or miR-210. In some embodiments, the exosome preparation includes exosomes enriched in miR-210.

In various embodiments, the oxygen concentration in the culture of cells that generate the exosome is 1-2%, 2-3%, 4-5%, 5-6%, 6-7%, 7-8%, 8-9%, 9-10%, 10-11%, 11-12%, 12-13%, 13-14%, 14-15%, 15-16%, 17-18%, or 18-19%. Preferably, the oxygen concentration is 2-8%, 3-7% oxygen, 4-6% oxygen, 4.5-5.5% oxygen. Exosomes generated from cells cultured in a lower oxygen concentration (e.g., 2-8% oxygen) differ in their RNA and protein constituents from exosomes generated from cells cultured in 20% oxygen.

In one embodiment, the protein content of the exosomes generated from cells cultured in 2-8% oxygen is higher than that of exosomes generated from cells cultured in 20% oxygen. In one embodiment, the total RNA content of the 5 exosomes generated from cells cultured in 2-8% oxygen is higher than that of exosomes generated from cells cultured in 20% oxygen. In one embodiment, the miR146A RNA content of the exosomes generated from cells cultured in 2-8% oxygen is higher than that of exosomes generated from cells cultured in 20% oxygen. In one embodiment, the miR-210 RNA content of the exosomes generated from cells cultured in 2-8% oxygen is higher than that of exosomes generated from cells cultured in 20% oxygen.

Harvesting Exosomes

Exosomes can be harvested from cell cultures by routine techniques. For example, when the cells reach confluency, they can be washed three times in 25 ml PBS, 30 ml of IMDM is added (without FBS) and put back in an incubator at a specified concentration of oxygen. After a period of time, the IMDM media can be removed and placed in 50 ml conical tubes. The media can be centrifuged at 3000×g for 15 minutes to eliminate cell debris. Media is separated into 10 ml fractions in 15 ml conical tubes and stored at −80° C.

Exosomes can be harvested after at least 1, 2, 3, 4, 5, 6, 7, 8, 9, 10, 11, 12, 13, 14, or 15 days in culture.

In some embodiments, exosomes are harvested every 2, 3, 4, or 5 days of culture.

Purifying Exosomes

Exosome preparations can be prepared by routine techniques in the art. In some embodiments, the preparation of exosomes includes centrifugation of the cells and/or media conditioned by the cells. In some embodiments, ultracentrifugation is used. In some embodiments, the preparation of exosomes from the population of cells is via size-exclusion filtration. In some embodiments, the preparation of exosomes from the population of cells includes use of discontinuous density gradients, immunoaffinity, ultrafiltration and/or high performance liquid chromatography (HPLC).

In some embodiments, differential ultracentrifugation is used, including using centrifugal force from at least 1000×g, 2000×g, 3000×g, 4000×g, 5000×g, 6000×g, 7000×g, 8000×g, or 9000×g, to 2000×g, 3000×g, 4000×g, 5000×g, 6000×g, 7000×g, 8000×g, 9000×g, 10,000×g, or larger to separate larger-sized particles from the exosomes derived from the cells.

In some embodiments, the preparation of exosomes from the population of cells includes use of filtration or ultrafiltration. In certain embodiments, a size exclusion membrane with different pore sizes is used. For example, a size exclusion membrane can include use of a filter with a pore size of at least 0.1, 0.5 μm, 1.0 μm, 2.5 μm, 5 μm, to 0.5 μm, 1.0 μm, 2.5 μm, 5 μm, or larger. In some embodiments, the pore size is about 0.2 μm. In some embodiments, filtration or ultrafiltration includes size exclusion ranging from 0.1 kDa, 0.5 kDa, 1 kDa, 2 kDa, 5 kDa, 10 kDa, 25 kDa, 50 kDa, 100 kDa, or 250 kDa to 0.5 kDa, 1 kDa, 2 kDa, 5 kDa, 10 kDa, 25 kDa, 50 kDa, 100 kDa, 250 kDa, 500 kDa, or more.

Preferably, isolated exosomes are filter sterilized with a 0.22 μm microbial exclusion filter. Preferably, exosomes are filtered using a 0.45 μm to remove cellular debris.

In various embodiments, such systems are used in combination with variable fluid flow systems. In other embodiments, the preparation of exosomes from the population of cells includes use of tangential flow filtration (TFF) systems are used purify and/or concentrate the exosome fractions. In other embodiments, the preparation of exosomes from the population of cells includes use of (HPLC) can also be used to purify exosomes to homogeneously sized particles. In various embodiments, density gradients as used, such as centrifugation in a sucrose density gradient or application of a discrete sugar cushion in preparation.

In other embodiments, the preparation of exosomes from the population of cells includes use of a precipitation reagent. For example, a precipitation reagent, such as EXOQUICK®, can be added to conditioned cell media to quickly and rapidly precipitate a population of exosomes. In some embodiments the preparation of exosomes from the population of cells includes use of volume-excluding polymers (e.g., polyethylene glycols (PEGs)). In another embodiment, the preparation of exosomes from the population of cells includes use of flow field-flow fractionation (FIFFF), an elution-based technique.

In some embodiments, PEG is used at a final concentration of 5%, 10%, 15%, or 20% to precipitate the exosomes. In some embodiments, the PEG has a molecular weight of about 4000, 6000, 8000, 10000, 12000, 15000, or 23000 Daltons. In some embodiments, the PEG has a molecular weight of about 4000-6000, 6000-8000, 8000-10000, 10000-12000, 12000-15000, or 15000-23000 Daltons.

In certain embodiments, the preparation of exosomes includes use of one or more capture agents to isolate one or more exosomes possessing specific biomarkers or containing particular biological molecules. In one embodiment, one or more capture agents include at least one antibody. For example, antibody immunoaffinity recognizing exosome-associated antigens is used to capture specific exosomes. In other embodiments, the at least one antibody are conjugated to a fixed surface, such as magnetic beads, chromatography matrices, plates or microfluidic devices, thereby allowing isolation of the specific exosome populations of interest.

In some embodiments, PEG is added to the exosome preparation after purification at a final concentration of 1-2%, 2-3%, 3-4%, 4-5% 5-6%, 6-7%, 7-8%, 8-9%, or 9-10%. In some embodiments, the PEG has a molecular weight of about 4000, 6000, 8000, 10000, 12000, 15000, or 23000 Daltons. In some embodiments, the PEG has a molecular weight of about 4000-6000, 6000-8000, 8000-10000, 10000-12000, 12000-15000, or 15000-23000 Daltons.

In some embodiments, an agent that causes aggregation of the exosomes is added to the exosome preparation prior to or after purification.

Analyzing Exosomes

Exosomes preparations from cell cultured in less than 20% oxygen can be analyzed. The exosomes can be compared to exosomes prepared from similar cells cultured in 20% oxygen. Whether the protein or RNA content of the exosomes generated from cells cultured in less than 20% oxygen (e.g., in 2-8% oxygen) is higher than that of exosomes generated from cells cultured in 20% oxygen can be determined using the techniques set forth herein or by other similar techniques.

The number and size of the exosomes can be quantitated, for example using Nanosight quantification.

The protein content of the exosomes can be analyzed using routine techniques to determining total protein levels or by using routine protein detection techniques (e.g., western blot) to determining the levels of specific proteins.

The RNA content of the exosomes can be analyzed using routine techniques to determining total RNA levels or by using routine nucleic acid detection techniques (e.g., PCR or probe hybridization) to determining the levels of specific RNAs. Preferred RNA are microRNAs, particularly miR-146A and miR-210 RNAs.

### EXAMPLES

### Example 1

### Exosome Preparation

Immediately upon receipt, hearts were grossly dissected and cut into biopsy-sized pieces of about 25 mg each (500 μm×500 μm×500 μm; though in some embodiments, other sizes are used), referred to as explants. Human hearts were cut using an automated tissue slicer (Zimmer® Dermatome) and automated tissue chopper (McMain™ Tissue Chopper, Ted Pella, Inc.) as previously described (see e.g. United States Patent US20150216905 A1). Explants were then processed as previously described (see e.g., Smith et al. 2007 and U.S. patent application Ser. No. 11/666,685, filed Apr. 21, 2007 and Ser. No. 13/412,051, filed Mar. 5, 2012, the entireties of each of which are incorporated by reference herein).

In order to generate allogeneic CDCs, explants were plated on CELLBIND® CeIISTACK® vessels (Corning Life Sciences). After 1-2 weeks, cellular outgrowth emerging from the explants became confluent. These explant derived cells (EDCs) were harvested using IX TrypLE™ (Invitrogen). EDCs were either cryopreserved as the master cell bank (MCB), and then cultured as cardiospheres (CSps), or placed immediately into CSp culture conditions. CSps were grown on UltraLow ® CellSTACK® vessels (Corning Life Sciences).

Allogeneic CDCs were grown by seeding CSps on fibronectin-coated Nunc* TripleFlasks (Thermo Scientific), and passaging when confluent. CDCs at varying passage number were seeded on to fibronectin-coated CellBind cellstacks and allowed to become confluent for exosome production. Upon confluence, media was exchanged to serum-free conditions (e.g. Iscove's Modified Dulbecco's Media with HEPES and L-glutamine). Cells were allowed to condition media for 5 or 15 days.

### Example 2

### Exosome Isolation

Exosomes were filtered using a 0.45 μm to remove cellular debris and then isolated by ultrafiltration based on size (2 kda to 30 kda), polyethylene glycol precipitation or Exoquick (SBI, Mountain View, Calif.). In certain situations, isolated exosomes were filter sterilized with a 0.22 μm microbial exclusion filter. Exosomes were formulated using several diafiltrations to replace the buffer to an acceptable infusion solution (e.g. Plasmalyte, Ringers's solutions).

### Example 3

### Exosome Analysis

Exosomal protein was assessed using DC assay (Bio-Rad, Hercules, Calif.). Exosome particle size and concentration was assessed using Brownian motion and the Nanosight tracking analysis (Malvern Instruments Ltd, Malvern UK). RNA was isolated using miRNeasy micro kit (Qiagen, Valencia, Calif.) and quantified using either the Nanodrop, Qubit or AATI fragment analyzer (Advance Analytics, Ankeny, Iowa). Reverse transcription and qPCR reactions were conducted using TaqMan miR probes (ThermoFisher Scientific, Grand Island, N.Y.).

We claim:

1. A method for generating exosomes comprising culturing cells in 2-8% oxygen for at least 5 days and harvesting exosomes from the cell culture.

2. The method of claim 1, wherein the cells are cultured for 5-15 days.

3. The method of claim 1, wherein the cells are cultured for 5-10 days.

4. The method of claim 1, wherein the cells are cultured for 10-15 days.

5. The method of claim 1, wherein the cells are cultured for at least 15 days.

6. The method of claim 1, wherein the cells are cardiosphere-derived cells (CDCs).

7. The method of claim 1, wherein the cells are passaged for at least 5 passages.

8. The method of claim 1, wherein the exosome preparation comprises less than 5% polyethylene glycol.

9. The method of claim 1, wherein the exosomes are purified using polyethylene glycol.

10. The method of claim 1, wherein the exosomes are purified using ultrafiltration.

11. The method of claim 1, wherein polyethylene glycol is added to the exosomes after purification.

12. The method of claim 1, wherein the cells are cultured in 3-7% oxygen.

13. The method of claim 1, wherein the cells are cultured in 4-6% oxygen.

14. The method of claim 1, wherein the cells are cultured in 4.5-5.5% oxygen.

15. An exosome preparation comprising at least 10⁶ exosomes generated from cells cultured in 2-8% oxygen for at least 5 days.

16. The exosome preparation of claim 15, wherein the miR-210 RNA and miR-146a content of the exosomes is higher than that of exosomes generated from cells cultured in 20% oxygen.

17. The exosome preparation of claim 15, wherein the cells have been cultured for 5-15 days.

18. The exosome preparation of claim 15, wherein the cells have been cultured for 5-10 days.

19. The exosome preparation of claim 15, wherein the cells have been cultured for 10-15 days.

20. The exosome preparation of claim 15, wherein the cells have been cultured for at least 15 days.

21. The exosome preparation of claim 15, wherein the cells are cardiosphere-derived cells (CDCs).

22. The exosome preparation of claim 15, wherein the cells have been passaged for at least 5 passages.

23. The exosome preparation of claim 15, wherein the exosome preparation comprises less than 5% polyethylene glycol.

24. The exosome preparation of claim 15, wherein the exosomes have been purified using polyethylene glycol.

25. The exosome preparation of claim 15, wherein the exosomes have been purified using ultrafiltration.

26. The exosome preparation of claim 15, wherein polyethylene glycol has been added to the exosomes after purification.

27. The exosome preparation of claim 15, wherein the cells have been cultured in 3-7% oxygen.

28. The exosome preparation of claim 15, wherein the cells have been cultured in 4-6% oxygen.

29. The exosome preparation of claim 15, wherein the cells have been cultured in 4.5-5.5% oxygen.
